(19) United States
(12) Patent Application Publication
(10) Pub. No.: US 2004/0034477 A1
(43) Pub. Date: Feb. 19, 2004

(54) Methods for modeling chromatographic variables

(76) Inventors: Michael McBrien, Etobicoke (CA); Eduard Kolovanov, Moscow (RU)

Correspondence Address: TESTA, HURWITZ & THIBEAULT, LLP, HIGH STREET TOWER, 125 HIGH STREET, BOSTON, MA, 02110, US

(21) Appl. No.: 10/290,111

(22) Filed: Nov. 7, 2002

Related U.S. Application Data
(60) Provisional application No. 60/404,439, filed on Aug. 19, 2002.

(51) Int. Cl. G06F 19/00; G01N 33/48; G01N 33/50; G01N 31/00

(52) U.S. Cl. 702/19; 702/22

(57) ABSTRACT

In one aspect the invention relates to a method of characterizing the suitability of chromatographic methods for use with a given compound of interest. This method typically includes providing structure information about the compound of interest. A structure similarity search based upon the structure information provided can also be performed. The structure similarity search is generally conducted within an application database. Evaluating chromatographic method parameters in response to structure similarities between the compound of interest and compounds present in the application database is also a component of this method. Relating the compound of interest to a suitable chromatographic method is yet another step in this method in various embodiments.

### CROSS-REFERENCE TO RELATED APPLICATIONS

This application claims the benefits of and priority to provisional U.S. Provisional Patent Application Serial No. 60/404,439, filed on Aug. 19, 2002, the disclosures of which are hereby incorporated herein by reference in their entirety.

### FIELD OF THE INVENTION

The present invention relates generally to the field of chromatography. In particular, the invention relates to chromatography modeling techniques and chromatographic method selection methodologies.

### BACKGROUND OF THE INVENTION

Methods for modeling chemical behavior and the impact of experimental parameters associated with that behavior continue to be an ongoing area of scientific interest and development. This is particularly true in the area of chromatography. The focus in this area has been fueled in part by the demand for information about unknown compounds in the medical, pharmaceutical, biological research, and industrial communities. As the methodologies and understanding of chromatographic approaches develop, better analytical and purification tools are made available to these communities.

In the experimental validation of combinatorial libraries, speed and high-throughput are often the key factors. For chromatographic separation or LCMS (Liquid Chromatography/Mass Spectrometry) of the newly synthesized compounds, generic chromatographic methods have been designed to accommodate the widest possible diversity of samples. However, when the sample is not suited to the chromatographic method, costly instrument downtime slows the analytical process, and whole plates or series of compounds are sometimes rejected.

Traditionally, a few chromatography methods have been used to assess a wide array of chemical samples. However, this often negatively impacts the timescale and accuracy associated with the experiment. For example, if samples are not retained by the column, the experiment must be rerun, consuming additional experimental and researcher time. Matching a non-optimal chromatography method with a particular compound of interest can also affect the accuracy of purity measurements. Further, since particularly on a preparative scale there is often a desired optimal elution volume, the choice of a given chromatography method may not result in a compound of interest eluting with this desired solvent volume. This adds to time costs as a result of the delay associated with the excess solvent evaporation time. Thus, the better matched a given chromatography method is to a given class of compounds, the more efficient the allocation of research efforts.

Finding ways to improve upon method selection and modeling compound elution behavior may ameliorate some of the undesirable affects that currently occur when using non-optimized methods. A need therefore exists to develop techniques for modeling the behavior of compounds of interest with respect to chromatographic methods. Developing such methods will optimize the value of data and information obtained from experimentation and chemical synthesis.

### SUMMARY OF THE INVENTION

The present invention relates to methods for modeling various parameters and variables which characterize the suitability of experimental chromatographic methods for use with a given compound of interest. Chromatography method and chromatographic method are used interchangeably throughout the application; as they refer to the same concepts and applied ideas as disclosed herein. Specifically, the invention relates to systems and methods for using known chromatographic data sets produced by different chromatographic methods to study new compounds. Further, different classes of chemical compounds are characterized by different physical and chemical characteristics prior to being investigated by various chromatographic methods. The suitability of various chromatographic measurement processes is associated with different diverse chemical species as a data set in various embodiments.

The invention is directed to using data generated by known chromatographic experimental results involving a training set to obtain predictive experimental information about how similar or dissimilar compounds will behave in various chromatographic experiments. A training set typically includes, but is not limited to chemical structures and retention times for a given chromatographic method. The techniques and core processes disclosed herein can be extended to all areas of chemical research employing chromatography-based methods.

In another aspect, the invention relates to using known chromatographic data obtained through standard chromatographic methods to gain predictive knowledge about future chromatographic trials of unknown compounds. In various aspects, the chromatography method used with a given compound to produce a given retention time and chromatography method parameters are linked in a generic application database. This linking of chromatography method, output results, and the chemical structure of the compound being studied enable predictive analysis of untested compounds in various embodiments. Similarly, the invention characterizes experimental data and generates predictive information about various chromatographic methods and related experimental parameters. These parameters include, but are not limited to the peak shape (peak symmetry and peak width) in a given chromatogram or its underlying data, the amount of solvent present in a given elution volume, impurity characteristics, retention time (t_{R}) and resolution among peaks within a given chromatogram. This list of parameters is not intended to be exhaustive as new parameters can be readily incorporated into the invention as they become desirable in a given experimental setup.

The chemical structure of known and hypothesized compounds, the method code (MC) for the particular type of chromatography used, and the retention time (t_{R}) (or retention factor, (k′)) within the chromatography system are other parameters used to obtain predictive chromatographic information in various embodiments. Any physicochemical characteristics which impact chromatographic behavior are parameters which may be used in various aspects of the invention. In some embodiments, these and the other aforementioned parameters can be used to generate user defined parameters which serve as quality terms for predicting which chromatographic method or methods should be used to run an experiment on an untested chemical compound. These user defined parameters can take the form of individual preferences regarding output data results, such as whether column retention time or peak resolution are most important to a user. Log P, pKa, Log D, molecular weight, molar refractivity, number of hydrogen donors and acceptors, polar surface area and molar volume can also be used to model retention behavior and for method selection in various embodiments of the invention. Log P is the hydrophobicity of a compound in its neutral form. PKa is a measure of the tendency of a molecule or ion to keep a proton, H⁺, at its ionization center(s). It is related to ionization capabilities of chemical species. Log D is the hydrophobicity of a compound, as it exists in aqueous solution at a given pH. If a compound is not present in solution wholly in neutral form, i.e., some ionization takes place, then a compound will be less hydrophobic than its Log P value suggests. For the most part, Log D is more relevant to reversed-phase chromatography than Log P.

Various mathematical models can be used to characterize a given chemical compound and/or chromatography method such as a linear model, a log model, a curve fit model, a hybrid model or other suitable mathematical model.

In one aspect of this invention, the predicted chromatographic response under one or more chromatographic methods for a set of potential compounds is compared to the experimental results for a sample. The set of potential compounds is then filtered based on the comparison of experimental and predicted results.

In another aspect of the invention, a software package performing the methods of the present invention is designed to advise if chromatography methods are viable, and select between available multiple methods. This application is also directed toward retention time and chromatographic method selection algorithms used to drive software based computational tools, as well as physical and chemical parameters used to model the chromatographic separation.

In one aspect the invention is a chromatographic software system that has been designed for “batchwise” evaluation of compounds to permit high-throughput method selection and accurate retention time prediction. A large number of varied structures injected under a limited number of chromatographic systems enable the software to characterize the methods based on predicted physicochemical parameters. This tool provides the ability to apply prediction as an added tool for verification of chemical structures, whether such structures are expected products or candidate impurities.

In some embodiments, the data processing device may implement the functionality of the methods of the present invention as software on a general purpose computer. In addition, such a program may set aside portions of a computer's random access memory to provide control logic that affects the hierarchical multivariate analysis, data preprocessing and the operations with and on the measured interference signals. In such an embodiment, the program is written in any one of a number of high-level languages, such as FORTRAN, PASCAL, DELPHI, C, C++, or BASIC. Further, the program in various embodiments is written in a script, macro, or functionality embedded in commercially available software, such as MATLAB or VISUAL BASIC. Additionally, the software in one embodiment is implemented in an assembly language directed to a microprocessor resident on a computer. The software may be embedded on an article of manufacture including, but not limited to, “computer-readable program means” such as a floppy disk, a hard disk, an optical disk, a magnetic tape, a PROM, an EPROM, or CD-ROM.

In one aspect, the invention relates to a method of evaluating the chromatographic characteristics of a compound of interest. The method includes providing an application database comprising a plurality of chemical chromatography method data, and known chemical structure information. Inputting chemical structure information for a compound of interest is also part of this method. The method typically includes performing a structure similarity search based upon the structure information provided for the compound of interest. The chromatography method data and known chemical structure information are typically related to the unknown compound of interest through a prediction equation. Solving the prediction equation to obtain compound of interest information is another feature of this method. In some embodiments, the chromatography method data includes predetermined target elution volumes. Method code parameters can also be included in the chromatography method data in various embodiments. The application database can include impurity information in various embodiments. Similarly, retention times can form part of the chromatography method data. Various other user defined parameters can also be incorporated in this aspect of the invention.

In another aspect the invention relates to a method of characterizing the suitability of chromatographic methods for use with a given compound of interest. This method typically includes providing structure information about the compound of interest. A structure similarity search based upon the structure information provided can also be performed. The structure similarity search is generally conducted within an application database. Evaluating chromatographic method parameters in response to structure similarities between the compound of interest and compounds present in the application database is also a component of this method. Relating the compound of interest to a suitable chromatographic method is yet another step in this method in various embodiments. In certain embodiments of this method, the effective pH associated with the chromatographic method parameters can be automatically modified in response to the compound of interest.

In another aspect the invention relates to a method for modeling retention times for a compound of interest. This method typically includes providing structure information about the compound of interest. Performing a structure similarity search based upon the structure information provided is also typically a feature of this method. The structure similarity search is typically conducted within an application database. Ordering retention time parameters in response to structure similarities between the compound of interest and compounds present in the application database can be carried out as part of this method. Predictive information relating the compound of interest to a predicted retention time can also be obtained through a prediction equation according to this method.

In yet another aspect the invention relates to a method of verifying the structure of a compound of interest. This method typically includes characterizing a data set of chromatographic methods for a plurality of known compounds. The data set includes at least one chromatographic parameter. Providing chromatography information about the compound of interest while obtaining chromatographic data for the compound of interest are also parts of this method. Comparing the chromatographic data for the compound of interest to the chromatographic data for similar compounds in the data set is another feature of this method. Evaluating the structure similarities of the compound of interest with known compounds in the data set in response to which chromatographic methods are suitable for both the compound of interest and the known compounds are yet another component of this method.

### BRIEF DESCRIPTION OF THE DRAWINGS

The invention is pointed out with particularity in the appended claims. The advantages of the invention described above, together with further advantages, may be better understood by referring to the following description taken in conjunction with the accompanying drawings. In the drawings, like reference characters generally refer to the same parts throughout the different views. The drawings are not necessarily to scale, emphasis instead generally being placed upon illustrating the principles of the invention.

FIG. 1 is an image of a computer based application illustrating some chromatographic related features of an embodiment of the invention;

FIG. 2 is a block diagram of various methods for evaluating chromatographic information according to an illustrative embodiment of the invention;

FIG. 3 is an image of a computer based application of the methods of the invention according to an illustrative embodiment;

FIGS. 4A-4G are images of a computer based application of the methods of the invention showing various features of an illustrative embodiment;

FIG. 5 is a graph illustrating the relationship of structural similarity and accuracy present in some embodiments of the invention; and

FIGS. 6A-6C are Venn diagrams illustrating the data filtering properties of using various chromatographic methods selected according to an illustrative embodiment of the invention.

### DESCRIPTION OF THE PREFERRED EMBODIMENTS

Embodiments of the present invention are described below. It is, however, expressly noted that the present invention is not limited to these embodiments, but rather the intention is that modifications that are apparent to the person skilled in the art and equivalents thereof are also included.

The invention relates, in part, to chromatographic methods, typically embodied in software. As used herein, chromatographic method denotes the complete instrument parameters and procedures associated with a particular chromatography experimental configuration. These instrument parameters typically include, but are not limited to the solvent system, column, gradient, temperature, and the dwell volume of the instrument. The methods of the invention enable selection of chromatography methods amongst a limited collection of such methods which are typically stored as part of an application database. Various aspects of the invention are suitable for use with a range of chromatography and analytical methods. For example, the application of the invention's techniques is suitable for use with the following non-exhaustive list of chromatography types: HPLC (High performance liquid chromatography), GC (Gas Chromatography), CE (Capillary Electrophoresis), high throughput solid phase extraction, and flash chromatography.

This method selection is based on the chemical structure(s) in the experimental sample undergoing chromatographic analysis. In addition, the chromatographic method selection facilitates selecting chromatography methods optimized for studying compounds of interest having particular chemical structures. These method selection techniques can be used in high-throughput, manual, or other suitable operational contexts. The invention also relates to determining retention times for various unknown compounds based on chromatographic data and chemical characteristics of known compounds of interest. The invention also relates to providing structural verification of compounds of interest based upon chromatographic parameter and chromatographic method information.

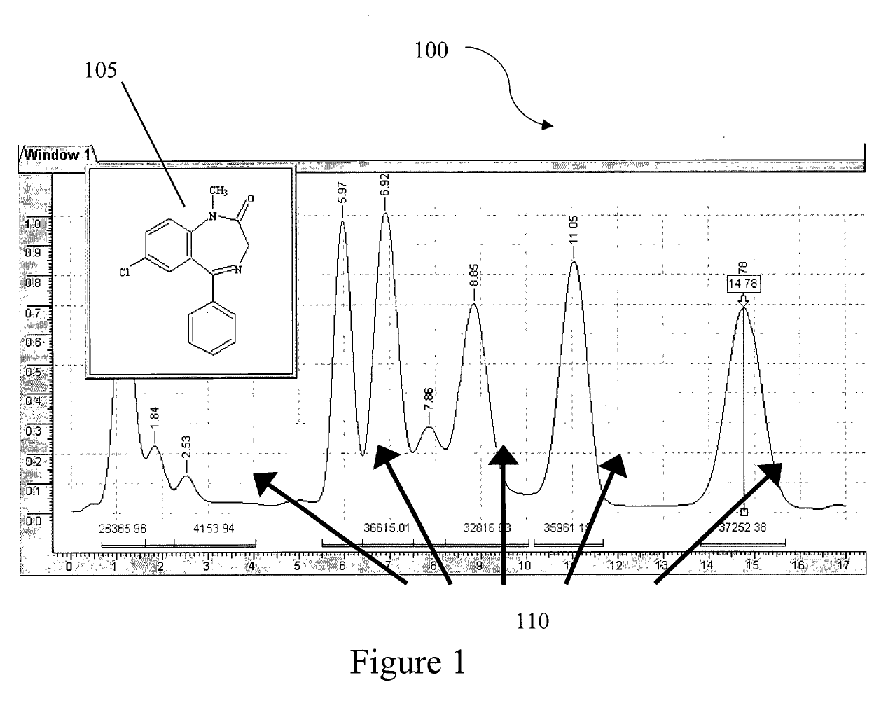

Referring to FIG. 1, an experimental data set in the form of chromatogram 100 is shown. A compound of interest 105 is also illustrated. The chromatogram 100 is for a compounds having some chemical properties in common with the compound of interest 105 in this embodiment. Relative peak intensity is shown along the vertical axis and retention time (t_{R}) is shown along the horizontal axis. In other embodiments, the retention factor k′ may be shown along the horizontal axis. The retention factor, (k′), is calculated as:

$$( \frac{{t}_{R} - {t}_{0}}{{t}_{0}} )$$

where (t_{R}) is the retention time and (t₀) is the dead time of the chromatography column. Thus, (t₀) is the time required by an inert compound to migrate from column inlet to column end without any retardation by the stationary phase.

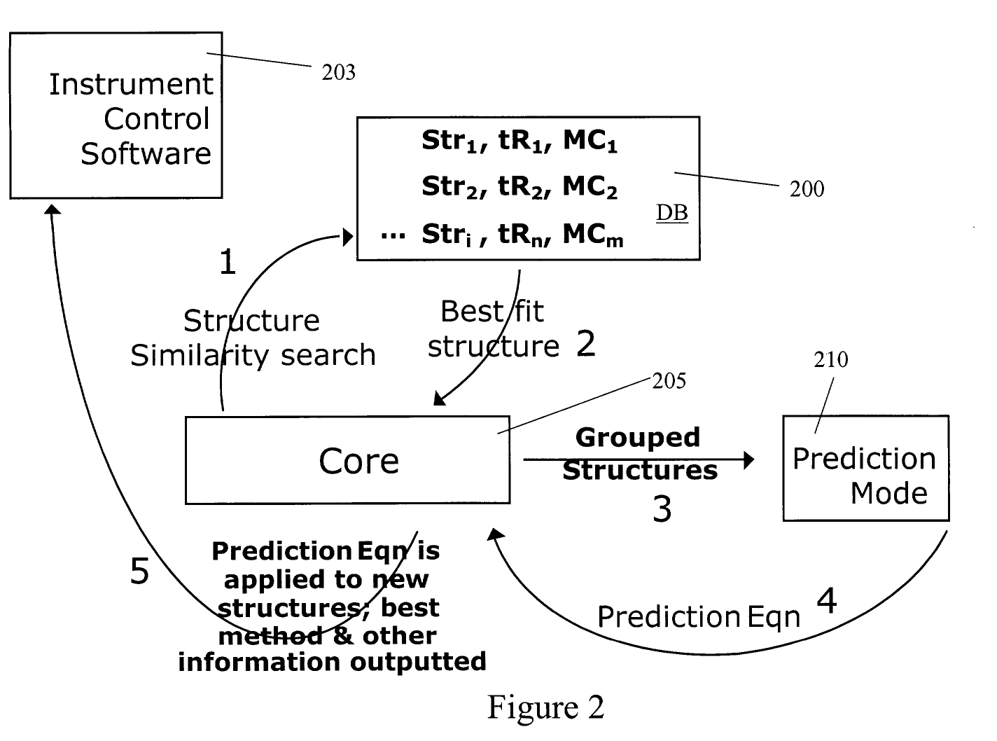

Given that the chromatogram 100 was selected with knowledge of the characteristics and structural features of the compound of interest 105 along with knowledge of the method parameters used to obtain the chromatogram 100, several avenues of inquiry arise. First, where will the compound 105 elute given the particular features of the chromatography method used? The arrows 110 shown in the diagram illustrate various places where the compound 105 may elute as a function of the chromatographic parameters and the structural features of the compound 105. The mechanics of modeling the compound's elution time are discussed in more detail below in the discussion of FIG. 2. FIG. 1 also raises a second question, how well suited is the given chromatography method for the particular compound 105? Answering these questions requires methodologies for chromatographic method selection and compound 105 retention prediction methods which represent different aspects of the invention. The method selection process is discussed in more detail with respect to FIG. 2 below.

Referring to FIG. 2, various components of an aspect of the invention and their interrelation with respect to chromatography method selection and retention time prediction are illustrated. In part, the invention generates prediction information regarding retention times and chromatographic method suitability based on an acquired knowledge base of known experimental chemical structures and retention times. This knowledge base typically takes the form of an application database, also sometimes referred to as a generic application database 200. The database 200 illustrated in FIG. 2 is shown to contain structural information indicated by STRᵢ, method code information (MCₘ), and retention time information (t_{Rn}) for various known compounds. Method Code information provides information about the parameters associated with a given chromatography method or experiment. The information depicted in FIG. 2, as resident within the database 200, is by no means complete or limiting. Information in the database 200 originates from instrument control software (ICS) 203 in various embodiments. The ICS typically controls the chromatography experiment and associated parameters, such as temperature or solvent flow, for example. The methods of the invention are described abstractly as the processing core 205 in FIG. 2. The processing core 205 directs and orchestrates the interaction of input information, data, and output results in various embodiments of the invention. In some preferred embodiments, the processing core 205 is a computer software program. The informational structures of the invention are open-format, therefore virtually any automated system can be compatibly configured. The invention provides additional links to the control software 203, such that chromatograms or simply the chromatogram's constituent data are automatically updated to the data base 200 once they are created. This allows for the core 205 to develop and “learn” as the database 200 evolves.

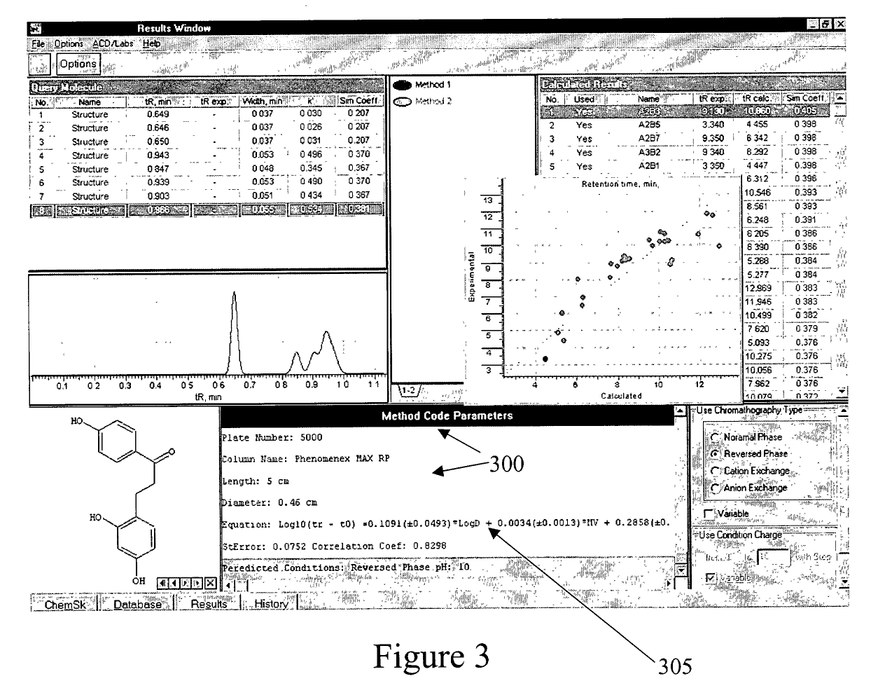

The contents of the generic application database 200 can vary between different embodiments of the invention and at different points in time for a given embodiment. Information about specific chemical compounds is incorporated in the application database 200 in addition to information about various chromatographic methods. In some embodiments, each entry in the database has an associated chemical structure, method code (MC), and an experimental retention time (t_{R}) or retention factor (k′). The Method Code links the chemical structure to the chromatographic method that was used to collect the chromatography data. An example of some method code parameters 300 used in one embodiment is shown in FIG. 3. Optionally, information about the chromatographic results, such as peak width, peak area, peak symmetry for example, obtained for a given compound are also associated with an entry in the database.

In other embodiments, additional information is included in the application database 200. This information can take the form of physicochemical parameters for the compounds; structure similarity indices; and/or predicted retention time. The physicochemical parameters for the compounds can include molecular weight, molar volume, Log P, Log D, polar surface area, hydrogen donors (HD), hydrogen acceptors (HA), and potentially more parameters and combinations thereof.

There are a large number of necessary parameters that are specific to the chromatographic method which are typically included in various embodiments of the application database. These can include column related parameters such as column name; column length, L in cm; column diameter, D in cm; column temperature; t₀, the dead time of the system; and combinations thereof. Additional chromatographic methods parameters can include the pH of the buffer; elution data (such as mobile phase, buffers, and gradient program for example); Flow rate, F in ml/min; Particle size, d in μm; and combinations thereof.

Again referring to FIG. 2, once the database 200 has been compiled, initialization calculations are directed by the processing core 205 of the invention. These calculations are performed in order to prepare the system to do calculations quickly. The majority of the calculations performed are predictions of retention times for compounds carried out as if the compound were not present in the database. The initialization steps, regulated and directed by the core 205, typically include initially indexing all compounds in the database with structure similarity indices, such as Dice coefficients for example. These indices can form the basis for the compound selection rules. These rules facilitate determining which database compounds share structural characteristics with a new compound of interest that has not undergone chromatographic analysis. Physicochemical values are also calculated for all compounds in the database. Retention times are calculated for each compound in the database as if that compound were not present.

Optionally, the invention can automatically modify the effective pH of the chromatographic model to improve the fit of the data. For example, it is well documented that the effective pH's of buffers change with addition of organic solvent. In addition, effective pKa's of compounds will change in the presence of these same organic solvents. With this in mind, the aqueous pKa for a given compound is not necessarily the best indicator of its ionization state in chromatographic conditions. One aspect of the invention relates to performing a correction for effective pH. The manner with which the invention performs this correction begins with the user typically defining the realm within which pH correction can be done. The processing core then examines the entire dataset for a given chromatographic method, predicting the retention times for each component as if it were not present in the dataset, and then compares the retention time to the experimental data. This series of steps is done for each of the potential pH values. The pH that gives the best overall agreement with experiment is the value that is used.

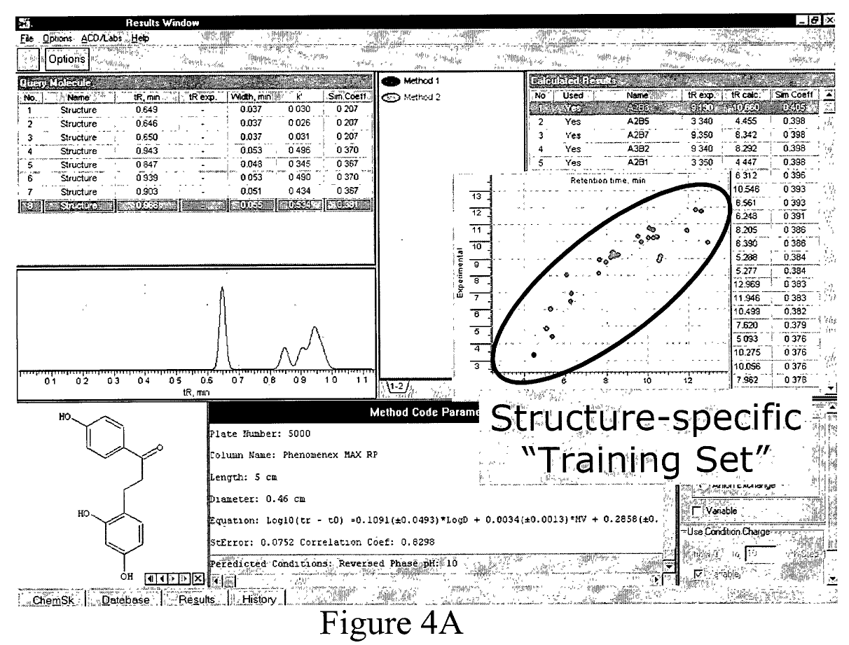

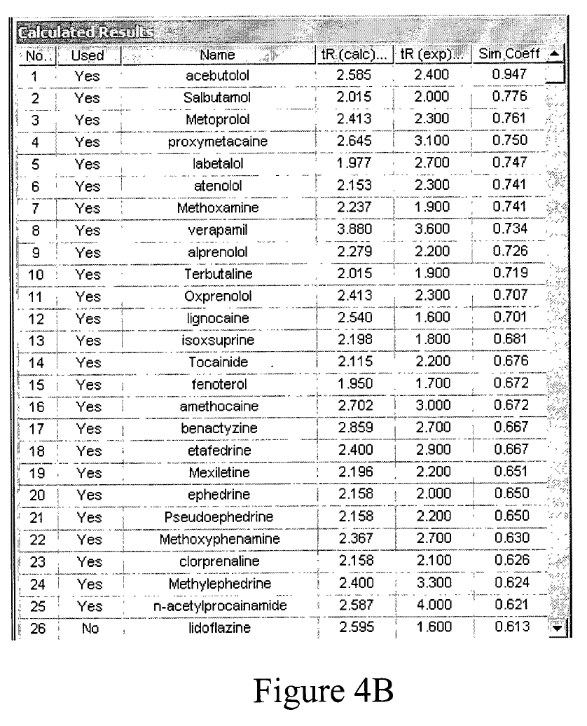

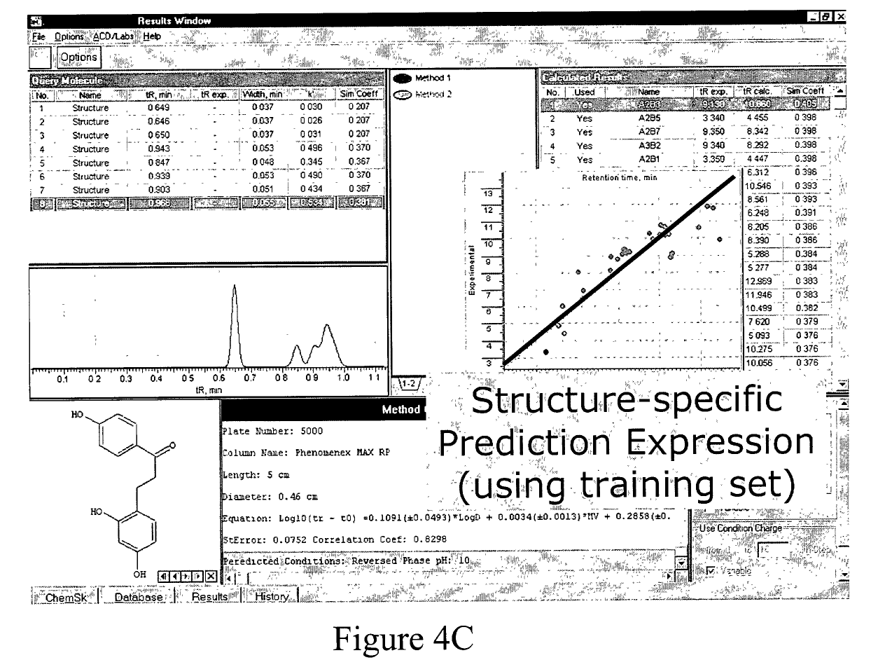

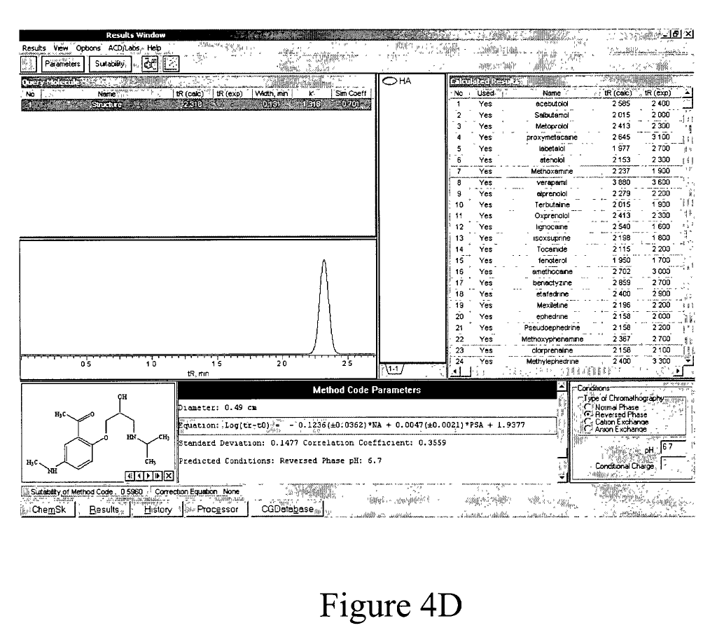

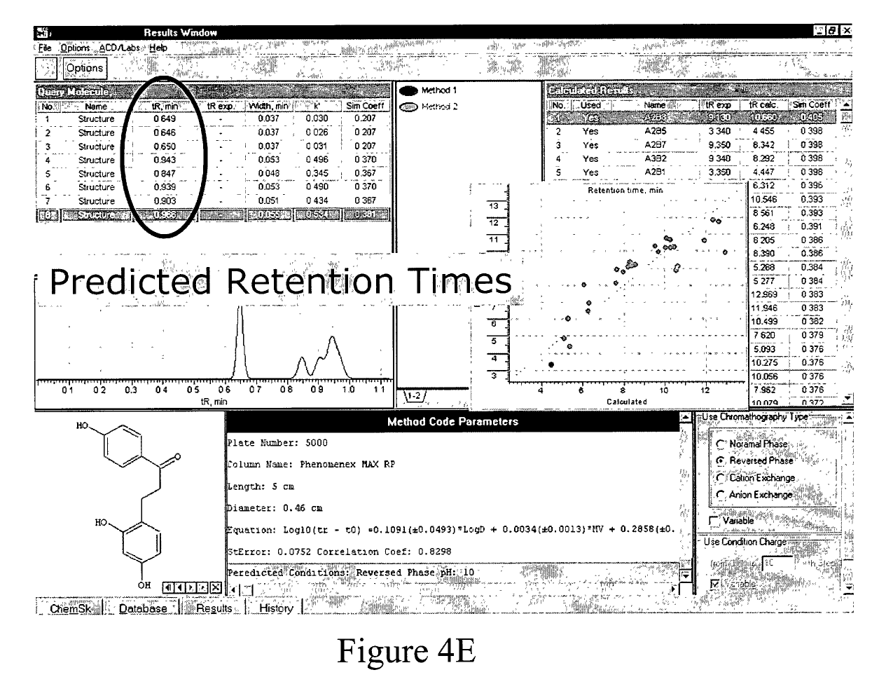

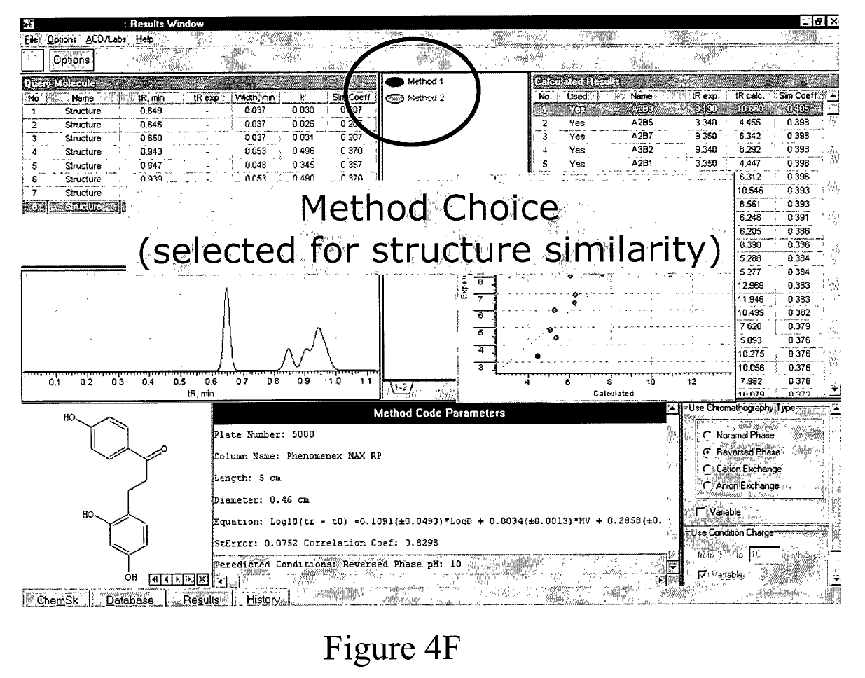

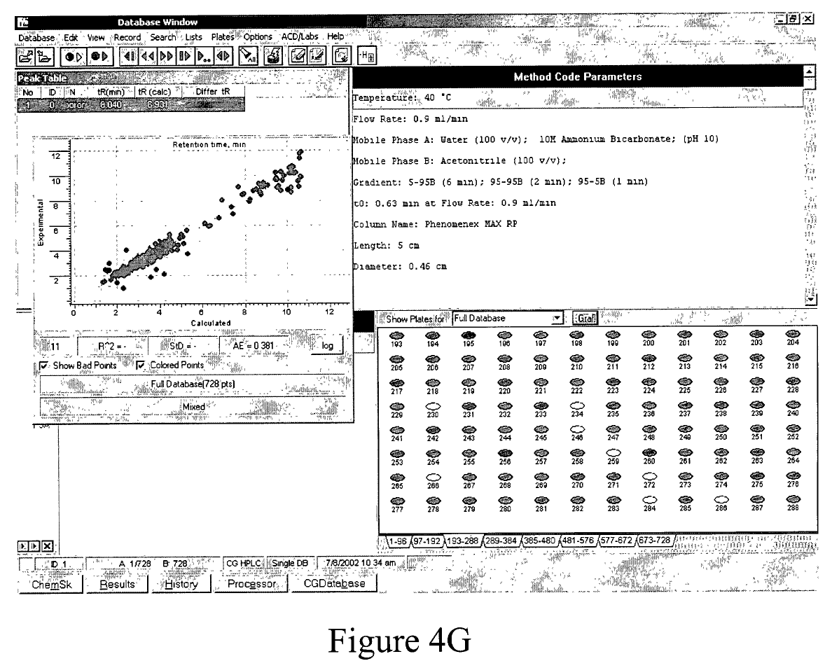

In one aspect, the methods of the invention calculate retention times for new compounds by the core 205 relating predicted physicochemical parameters of the archived compounds to their elution times. The accuracy of this model is greatly enhanced by the employment of structure similarity searches to choose the most relevant compounds to the ones in question. Prediction of retention times for a given structure is thus done in several steps. These occur after the assembly or provision of an application database and the performance of the initialization calculations. Initially, the core 205 uses inputs about the compound of interest to search the database to find the most relevant compounds. This search is generally a structure similarity search, which is discussed in more detail below. This narrows the application database to a reduced data set of relevant information. Retention times and other parameters of other compounds injected under a given chromatographic method are used as a “training data set”. An example of such a training set is shown in FIG. 4A. Another example of a “training set”, set of structures and their retention times under a given set of experimental conditions, is shown in FIG. 4B. The structures are selected for their similarity to the test structure(s). The user option has been set to search for the “25 most similar compounds” in the example shown in FIG. 4B. With respect to the training data set, the compounds that are used as the basis for prediction are a subset of the complete database. The number of molecules selected is a function of average similarity to the test molecule (compound of interest) and their similarity to each other as discussed below.

The elution times of new untested compounds are predicted in relation to the training set. Using standard methods, compounds from many chromatographic experiments can be grouped. In some embodiments of the invention there are specific factors which relate to prediction of (t_{R}s). In the context of reversed phase chromatography, hydrophobicity (Log D), molecular weight, molar volume, molar refractivity, and other relevant parameters impact retention time. These factors are typically included in modeling the retention times in reversed phase chromatography experiments.

The core 205 and associated methods of the invention employ different approaches for structure similarity searching. A structure similarity search is a generic term describing various methods of fragmenting molecules and ranking similarities based on the number of common molecular fragments. The relationship between structure similarity and accuracy is tied to data set characteristics. For each compound in the database, structures are typically sorted according to similarities.

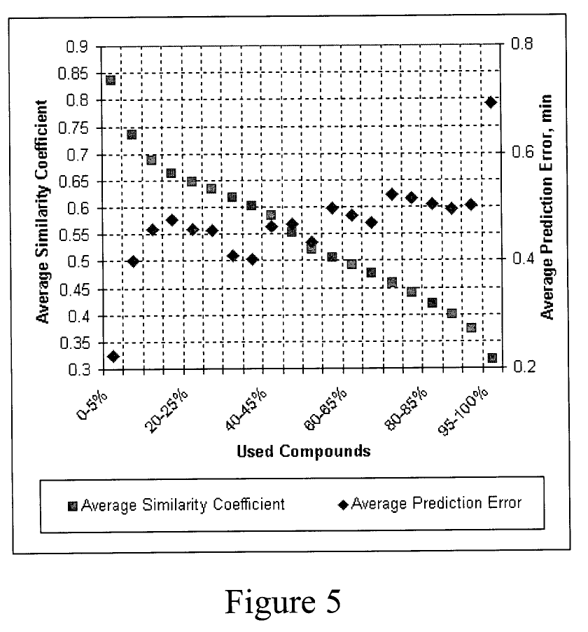

In particular, the accuracy of prediction increases as similarities between the compounds of interest and those forming the training set increase. This point is illustrated by the graph in FIG. 5, which shows that the average error goes from 8 to 16% as similarity goes from 0.85 to 0.4. Structural similarity can vary between 1 and 0. Each compound graphed used some portion of the rest of the compounds as training set. The graph in FIG. 5 is based upon testing some of the methods of the invention on 654 compounds. Training sets of 32 or 33 were chosen in groups. Experimental (t_{R}) was compared to predicted (t_{R}). This showing of average error being tied to structural similarity validates aspects of the invention's operation.

Referring back to FIG. 2, by binning similar structures in the database 200, the core 205 is ultimately able to develop a better method choice, find compounds with similar retention times, and select a reduced data set of compounds with similar retention mechanism. All of these factors lead to more accurate predications in various aspects of the invention. The best fit results of the similarity search are obtained from the generic application database 200 for use by the methods and operational techniques of the core 205.

Databases of molecular structures play an increasingly important role in modern chemical research. Substructure searching has proved to be a valuable tool for accessing these databases, however this type of search has several limitations that arise from the requirement that a database structure must contain the entire query substructure if it is to be retrieved, which implies that the user who is posing a database query must already have formed a fairly clear view of the types of structure that should be retrieved. The user also has very little control over the size of the output that is produced by a particular query substructure. Thus, the specification of a broadly defined query can result in the retrieval of many thousands of compounds from a chemical database; alternatively, an initial query may prove to be more specific, retrieving very few, or even no structures. In either case, it may be necessary to reformulate the query one or more times before an appropriate volume of output is available for subsequent analysis.

These characteristics of substructure searching have led to the development of the alternative, and complementary, access mechanism known as similarity searching. A query here generally involves the specification of an entire molecule, the target structure, rather than the molecule fragment that is required for substructure searching. The target is characterized by one or more structural descriptors, and this set is compared with the corresponding set of descriptors for each of the molecules in the database. These comparisons enable the calculation of measure of similarity between the target structure and each of the database structures, and the latter are then sorted into order of decreasing similarity with the target. The output from the search is a ranked list in which the structures that are deemed to be the most similar to the target structure, the nearest neighbors, are located at the top of the list. These neighbors form the initial output of the search and will be those that have the greatest probability of being of interest to the user, given an appropriate measure of intermolecular structural similarity.

The principal challenge is quantifying the similarity or degree of structural resemblance between the target structure and each of the structures in the database that forms the basis of the search. The similarity coefficient provides a quantitative measure of structural relatedness between a pair of structural representations. The similarity coefficient determines a numerical measure of similarity (or conversely, the distance) between two objects, each characterized by a common set of attributes. A review of the coefficients that have found widespread use in chemical information systems is useful for illustrating an embodiment of the invention.

Each structure is represented as a binary vector containing (n) attributes. Let A be the target structure, and let B correspond to any structure resident within the database. Further, let X_{A}={x_{1A}, x_{2A} . . . , x_{jA}, . . . , x_{nA}} and X_{B}={x_{1B}, x_{2B} . . . , x_{jB}, . . . , x_{nB}} be binary vectors describing the structures A and B respectively. The vectors are binary in the sense that the attributes of the respective vectors A and B are either 0 or 1. If the structure object number (i) is present in A, x_{iA} equals one. If the structure object number (i) is absent in structure A, x_{iA} will equal zero. Structure objects are generated automatically depending on database structure types.

Let ^{S}AB and ^{D}AB be respectively similarity and distance between the structures A and B Let.

$$a = {\sum }_{j - 1}^{n} {x}_{j A} b = {\sum }_{j - 1}^{n} {x}_{j B} c = {\sum }_{j - 1}^{n} {x}_{j A} {x}_{j B}$$

$$d = {\sum }_{j - 1}^{n} ( 1 - {x}_{j A} - {x}_{j B} + {x}_{j A} {x}_{j B} )$$

A minimum coefficient list is a set of compounds selected based upon a common minimum similarity value determined for a given user directed query. Thus a minimum coefficient list associated with a similarity coefficient of 0.80 would contain a group of compounds that each were 80% similar to particular queried compound of interest. Let m be a similarity value associated with a particular minimum coefficient list. If we let S_{AB} be the similarity between objects A and B, we will also let a and b be the number of “bits” that are “on” in molecules A and B respectively and let c be the number of “bits” that are “on” in both molecules A and B. The following table describes how the processing core 205 typically searches records by similarity:

| Coefficient | Parameter | Condition |
|---|---|---|
| Tanimoto | ${S}_{A , B} = \frac{c}{a + b - 2 c}$ | ${S}_{A , B} ⩾ m$ |
| Dice | ${S}_{A , B} = \frac{2 c}{a + b}$ | ${S}_{A , B} ⩾ m$ |
| Cosine | ${S}_{A , B} = \frac{c}{\sqrt{ab}}$ | ${S}_{A , B} ⩾ m$ |
| Based on Hamming Distance | ${D}_{A , B} = 1 - \frac{a + b - 2 c}{n}$ | ${D}_{A , B} ⩾ m$ |
| Based on Euclidean Distance | ${D}_{A , B} = 1 - \frac{\sqrt{a + b - 2 c}}{n}$ | ${D}_{A , B} ⩾ m$ |

If the prescribed conditions are fulfilled, the database record containing the structure B will be shown as a result of the search. The core 205 directs the display of records in descending order based upon the similarity coefficients.

These concepts are discussed in more detail in the article “Chemical Similarity Searching” by Willet, Peter, John M. Barnard, and Geoffrey M. Downs J. Chem. Inf. Sci. 1998, 38, 983-996. The contents of the “Chemical Similarity Searching” article are herein incorporated by reference in their entirety.

The similarity search algorithm used in these embodiments can be any of the Dice, Tanimoto, or other published algorithms. In a preferred embodiment, Dice coefficients are used in the structure similarity features of the invention. The Dice similarity indices are used in various embodiments to compare associations among chemical structures as discussed above. Although this discussion of structure similarity searching can be used in some embodiments, it is not intended to limit the invention to one searching methodology. Other suitable searching methods and algorithms can be developed for use in other embodiments.

Referring again to FIG. 2, during the initialization calculation directed by the core 205 various predicted chromatographic parameters are generated with respect to compounds possessing structural characteristics. After a subset of compounds has been identified during the search, these predicted physicochemical parameters are processed according to a suitable prediction algorithm. This prediction mode 210 is initiated and controlled by the processing core 205. The prediction algorithm develops a prediction equation for the method using the reduced data set. An illustrative example of a feature of the predictive mode 210 is shown for one embodiment in FIG. 4C.

The predictive mode of the invention relates to various features and embodiments of the invention. One of these features relates to predicting the retention times of compounds in a given chromatographic system. Predicted retention times, in turn, are used to evaluate the applicability of a given chromatography method to a given chemical sample. Such predictions are typically done by predicting physicochemical parameters for compounds (the training set) archived in the database. Those database archived compounds found to be most similar to the given test compound(s) are selected from the database. The experimental parameters associated with the selected archived compounds are related to their experimental retention times to generate a “prediction equation”. Once this is done, physicochemical parameters are generated for the test compound(s), and the prediction equation is used to predict corresponding retention times. The steps associated with generating one or more predictions equations can be referred to as a prediction algorithm.

One aspect of the invention is directed to creating a fit between structure and retention time as a function of physicochemical parameters including partition coefficient (Log D [hydrophobicity of a compound, as it exists in aqueous solution at a given pH] or Log P [hydrophobicity of a compound in its neutral form]), molecular weight (MW), Molar Refractivity (MR), Molar Volume (MV), number of proton donors (ND), number of proton acceptors (NA), polar surface area (PSA), and boiling point (BP). Different parameters are often used with different chromatography methods to develop correlations for formulating suitable prediction equations. The parameters typically used for Reversed Phase (RP) HPLC include, but are not limited to: Log P, Log D, MW, MV, MR, PSA, HA, HD, and combinations thereof. The parameters typically used for Ion-exchange (IE) HPLC include, but are not limited to: Log P, Log D′, MW, MV, MR, PSA, HA, HD and combinations thereof. Log D′ is the Log D corrected according to the ion-exchange character of the separation. The parameters typically used for Normal Phase (NP) HPLC include, but are not limited to: Log P, MW, MV, MR, PSA, HA, HD and combinations thereof. The parameters typically used for Gas Chromatography (GC): include, but are not limited to: BP, Log P, MW, MV, MR, PSA, HA, HD and combinations thereof.

An expression for the k′ (capacity factor) of a component at a given pH can be developed accordingly for the various chromatography methods discussed previously. This is shown below (Eq. 1-Eq. 4) for a non-exhaustive list of chromatography methods. In equations 1-4, listed below the “I” parameter is an experimentally determined function or constant. Similarly, the A, B, C, D, E, F, G, and H prediction equation parameters shown below can assume functional or constant values in various embodiments. In those embodiments, wherein the prediction equation parameters are constants, they can have negative or positive values.

NP: Log(k′)=A(Log P)+B(MR)+C(MW)+D(MV)+E(PSA)+F(NA)+G(ND)++I (Eq. 1)

RP: Log(k′)=A(Log D)+B(Log P)+C(MR)+D(MW)+E(MV)+F(PSA)+G(NA)+H(ND)+I (Eq. 2)

IE: Log(k′)=A(Log D′)+B(Log P)+C(MR)+D(MW)+E(MV)+F(PSA)+G(NA)+H(ND)+I (Eq. 3)

GC: Log(k′)=A(BP)+B(Log P)+C(MR)+D(MW)+E(MV)+F(PSA)+G(NA)+H(ND)+I (Eq. 4)

Optionally, any of the terms in a prediction equation can be omitted from the expression based on the user preferences. Thus, although only four exemplary prediction equations are shown the type and number of prediction equations is vast. An example of a prediction equation and some of these features of the invention is shown in FIG. 4D. In other embodiments, known k′ values in conjunction with other known parameters can be used to predict other compound parameters.

These equations are an approximation, and other factors contribute to the capacity factor (k′) for a given compound in a given chromatography system. The accuracy of this approach is linked to the use of similar compounds as the training set. The predicted physicochemical values for the test compound(s) are input to the prediction equation, which then gives the expected retention factor for the compound. The retention factor k′ is then converted into a retention time output.

The arguments of the prediction equation are determined in part by the optional settings and in part by the behavior of the separation (an algorithm with elements of principal component analysis determines which of the arguments are relevant to the situation). One logarithmic compound specific embodiment of a prediction equation 305 is shown in FIG. 3. In order to predict retention times for new compounds, the core 205 calculates the relevant physicochemical parameters and inserts them into the prediction equation. Thus, by (1) comparing the known structural characteristics of a compound of interest to the information contained within the database and (2) using various predictive algorithms and equations, the compound's retention time in a particular chromatographic experiment can be accurately modeled. One embodiment of the invention showing predicted retention times is shown in FIG. 4E.

The Log D/Log P/pKa parameters, in particular, are effective in the prediction of retention times in virtually all kinds of chromatography. Thus some of the aspects of the invention relating to these parameters will be described in some detail. Estimating a compound's partition coefficient (Log D) is typically performed by combining predictions for Log P and pKa. Log P, the partition coefficient, is computed according to a fragment based approach. This means that the compound under investigation is broken into constituent molecular fragments, and each such fragment is assigned a value. These values are summed for all constituent pieces to construct the Log P prediction. Several factors are relevant to this approach: 1) the choice of principle applied to determine which molecular substructures are the correct fragments to use; 2) the corrections that need to be applied for through-space and through-bond interactions between these molecular fragments; and 3) the means by which the contribution value is determined.

In one embodiment of the invention the principle applied to determine which fragments to use is the “Principle of Insulating Carbons”. This principle results in a molecule being inspected for carbon atoms that, if removed, would not alter the overall electronic structure of the compound. These are the insulating carbons, while the fragments are the remaining substructures. Thus the molecular fragments of interest are what remains once all such insulating carbons are removed. Each fragment may, in certain molecules have some residual interactions with other fragments, notwithstanding this principle of insulating carbons. However, these cases can be classified, and the magnitude of these interactions can be calibrated. This classification and calibration addresses some of the corrections which can be performed. All values for fragmental contributions and interactions are typically assessed by analyzing thousands of compounds, and employing statistical regression techniques that optimize the predictive quality of the algorithm and its statistical significance.

The pKa, the ionization constant is computed in a different fashion. Ionization centers are recognized in the chemical structure by paying attention to the structural patterns around Nitrogen, Oxygen, Sulfur and some Carbon atoms. For each such ionization center, a Hammett equation is constructed that reflects how the pKa is modulated by the chemical structure elements (substituents) surrounding the ionization center for the specific ionic form of the compound before de-protonation. In the Hammett equation, the modulation effect (electron withdrawal) is quantified by a variable Sigma. The pKa can be modeled based upon this equation.

Given a prediction of all of a molecules pKa's and the Log P's for each ionic form theoretically achievable, Log D is computed by solving the multi-equilibrium system of equations. In cases of several ionic forms, this equation is often simplified to yield an approximate solution where it is possible to do so without introducing additional error. It should be noted that these equilibria are highly pH dependent and the general solution of these equations yields the curve Log D vs. pH. Various other parameters can be modeled using techniques similar to those disclosed herein or known to those of ordinary skill in the art.

Although predicting retention times is one aspect of the invention, selection of chromatographic methods is yet another. The process for selecting chromatography methods, according to one embodiment, is similar to the process outlined in FIG. 2. A compound of interest is compared to the database 200 through a structure similarity search and the chromatography methods associated with the compounds are selected based upon the degree of structural similarity between known compounds and the compound of interest. The decision as to what makes a desirable chromatographic method also depends on the interplay between the user priorities and the characteristics of the compound of interest. In preparative scale chromatography, for example, the most important priorities are usually peak shape and width and elution time. If peaks are narrow and symmetrical, it is easy to collect all of the material in a small volume of solvent. If the compound elutes in the correct time frame and the solvent can easily be evaporated from the resulting sample then this method can be recommended for the preparative separation. In virtually any application resolution from impurities is important. The chances of choosing a method that will give good resolution can be enhanced by three factors: similar structures having been archived with a given method implies a good chance of success; a reasonably long retention time implies that there is a greater chance of resolution from impurities; and any known impurities can also be predicted, and resolution and retention times of the impurities modeled and factored into the analysis. One embodiment of the invention showing chromatographic method selection results is shown in FIG. 4F.

The priorities of the user serve as inputs into the operational elements of the invention, which form the processing core 205. If impurities are input, resolution from them can be prioritized. Methods that do not give peak resolution are de-prioritized. Retention times can be specified as “hard” and “soft” requirements. For example, users can specify required compound elution times of between 2 and 6 minutes, preferred elution between 4 and 6 minutes. In this example, methods that will not elute between 2 and 6 minutes are rejected, but the methods outside the 4-6 range will only be rejected if another method appears better. These requirements are individually customizable for each method and can be tailored to comply with any output requirements. Such chromatographic parameters as required, suitable minimal k′ as required and suitable asymmetry, etc. also may be employed by the user as priority parameters. If the user has not specified priority parameters for some reason, these parameters will be calculated by default in correspondence with USP 24 (Validation of compendia methods). Available parameters and their default values are different for isocratic and gradient experiments. If two methods score equally based upon user needs, average structure similarity of the known compounds retrieved from the database 200 is the final arbiter. The assumption is that as long as methods that are in the database 200 have been successful for the compounds to which they have been linked, the more similar the compounds, the more likely the success of the predicted chromatography method with the compound of interest.

The methods of the invention can also be used to assist with structure verification objectives. High-throughput structure verification is generally performed with limited amounts of data. Often, structures are verified based only on molecular weight data gleaned from mass spectrometry. Inevitably, this results in errors; even accurate mass data will only verify structural formula, not structure. An added clue is available in the form of the experimental retention time of the compound. After collection of the data, various features of the invention can direct the display of the compounds that have retention times outside the expected range. For this purpose, the initialization calculation becomes very important. Since an analysis of performance against a large data set has been conducted as shown by FIG. 5, accuracy as a function of structure similarity is modeled. The user thus has the ability to choose the displays (yes/no/maybe) as a function of error percent or % probability that a compound could elute at the experimental retention time. For example, the user could specify that compounds that would elute at a given time only 5% of the time or less be keyed yellow, but compounds that would elute at the given time 1% of the time or less be keyed red. This gives the chromatographer more evidence of success or failure in synthesizing the compound of choice. One embodiment of the invention showing structural verification results is shown in FIG. 4G.

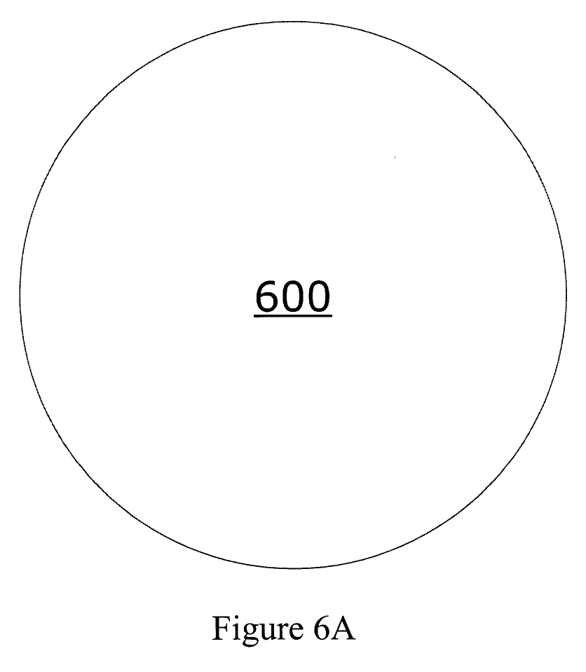

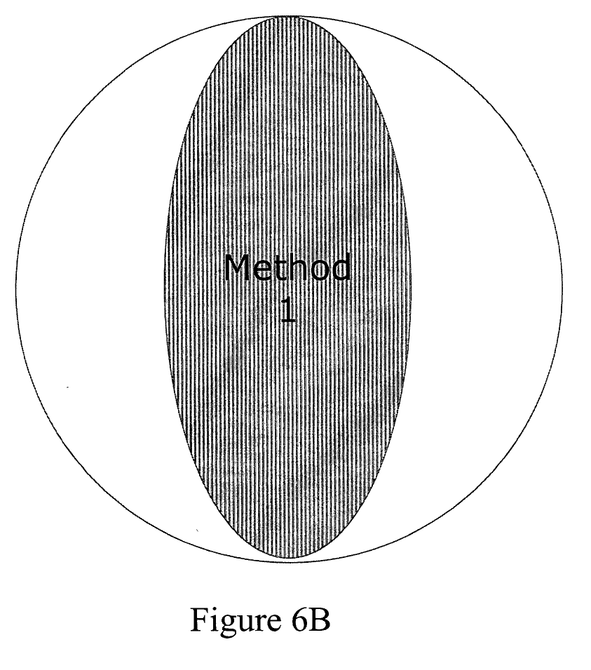

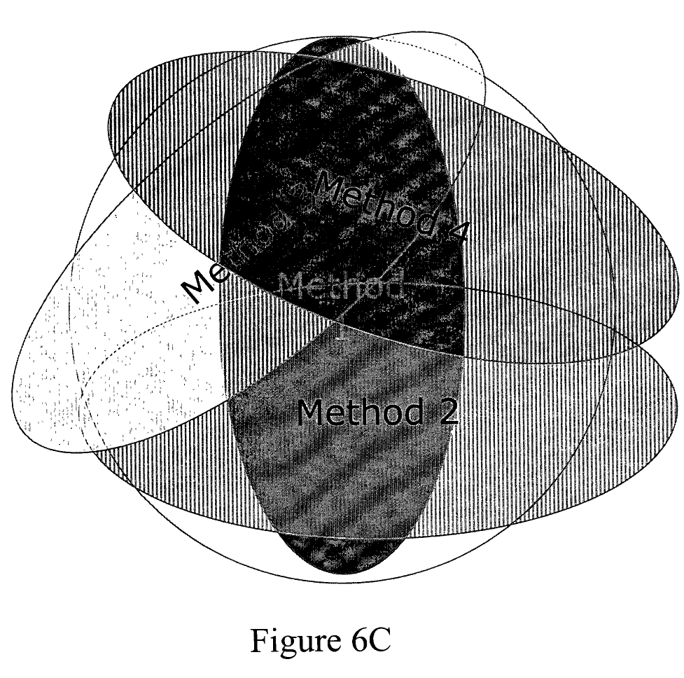

Retention times generally cannot be used for structure elucidation. However, with accurate prediction,(t_{R}s) can be used to filter candidate structures down to manageable numbers without the need for additional experiments based on comparison to experimental retention time. In many cases with an unknown compound, a researcher may be faced with a large number of potential structures that fit the collected experimental data. This large number of potential compounds can be reduced based on any standard chromatographic experiments that may have been performed on the compound. Referring to FIG. 6A, initially a set of anticipated compounds is characterized or delineated as shown by the generalized Venn diagram shown. This representation is included to further illustrate the structure verification aspect of the invention. Again by chromatography method it is worth noting that this does not simply refer to a type of chromatography, such as HPLC for example, but rather includes how the experiment was performed and all or a subset of all the parameters associated with that experimental run. Incrementally in FIG. 6C, the overlap of additional chromatography methods (Methods 2, 3 and 4) and particular anticipated compounds 600 are shown. Thus, results from multiple chromatographic experiments can be used to narrow down lists of candidate compounds in one aspect of the invention. For example, if one chromatographic method can exclude ⅔ of candidate structures (reducing a list by 67%), four chromatographic methods can theoretically exclude 80/81 of candidate structures, reducing a list by approximately 99%. Lists of candidate structures can be created by metabolite prediction software, reaction prediction software, structure elucidation software, manual prediction or combinations thereof.

Researchers can collect new data to form the basis of the retention time screen, or they can use data that has been previously collected. Given a reasonable level of certainty as to the elution time of compounds under a given method, the archived experimental t_{R} can serve as the basis for structure verification. Thus, even months after the original method development work has been completed, the experiments that were used can help to study the impurities or anticipated compounds involved. This structural verification feature of the invention can be combined with the chromatography selection methodologies and the retention time calculations in various embodiments to improve experimental results.

While the present invention has been described in terms of certain exemplary preferred embodiments, it will be readily understood and appreciated by one of ordinary skill in the art that it is not so limited and that many additions, deletions and modifications to the preferred embodiments may be made within the scope of the invention as hereinafter claimed. Accordingly, the scope of the invention is limited only by the scope of the appended claims.

What is claimed is:

1. A method of evaluating the chromatographic characteristics of a compound of interest, the method comprising the steps of:
providing an application database comprising a plurality of chemical chromatography method data, and known chemical structure information;
inputting chemical structure information for a compound of interest;
performing a structure similarity search based upon the structure information provided for the compound of interest;
relating the chromatography method data and known chemical structure information to the unknown compound of interest through a prediction equation; and
solving the prediction equation to obtain compound of interest information.

2. The method of claim 1 wherein the chromatography method data includes predetermined target elution volumes.

3. The method of claim 1 wherein the chromatography method data includes method code parameters.

4. The method of claim 1 wherein the application database further includes impurity information.

5. The method of claim 1 wherein the chromatography method data includes retention times.

6. The method of claim 1 wherein the database includes user defined parameters.

7. The method of claim 1 comprising the step of determining similarity coefficients for compounds archived in the database.

8. The method of claim 1 comprising the step of determining retention times for compounds archived in the database.

9. The method of claim 1 wherein the application database contains at least one of Log P data, pKa data, Log D data, molecular weight data, molar refractivity data, number of compound hydrogen donors and acceptors data, polar surface area data, boiling point data, or molar volume data.

10. The method of claim 1 further comprising the step of automatically modifying the effective pH associated with the chromatography method data.

11. A method of characterizing the suitability of chromatographic methods for use with a given compound of interest, the method comprising the steps of:
providing structure information about the compound of interest;
performing a structure similarity search based upon the structure information provided, wherein the structure similarity search is conducted within an application database;
evaluating chromatographic method parameters in response to structure similarities between the compound of interest and compounds present in the application database; and
relating the compound of interest to a suitable chromatographic method.

12. The method of claim 11 further comprising the step of automatically modifying the effective pH associated with the chromatographic method parameters in response to the compound of interest.

13. The method of claim 11 wherein the suitability of the chromatographic methods is determined in response to experimental retention times.

14. The method of claim 11 wherein the application database contains at least one of Log P data, pKa data, Log D data, molecular weight data, molar refractivity data, number of compound hydrogen donors and acceptors data, polar surface area data, boiling point data, or molar volume data.

15. A method for modeling retention times for a compound of interest, said method comprising the steps of:
providing structure information about the compound of interest;
performing a structure similarity search based upon the structure information provided, wherein the structure similarity search is conducted within an application database;
ordering retention time parameters in response to structure similarities between the compound of interest and compounds present in the application database; and
generating predictive information relating the compound of interest to a predicted retention time through a prediction equation.

16. The method of claim 15 wherein the prediction equation is determined in response to the chromatography method used.

17. A method of verifying the structure of a compound of interest, the method comprising the steps of:
characterizing a data set of chromatographic methods for a plurality of known compounds, wherein the data set includes at least one chromatographic parameter;
providing chromatography information about the compound of interest;
obtaining chromatographic data for the compound of interest;
comparing the chromatographic data for the compound of interest to the chromatographic data for similar compounds in the data set; and
evaluating the structure similarities of the compound of interest with known compounds in the data set in response to which chromatographic methods are suitable for both the compound of interest and the known compounds.

18. The method of claim 17 wherein the chromatography data provided is an experimental retention time for the compound of interest.

19. The method of claim 17 comprising the step of excluding known compounds having retention times substantially different than the retention time of the compound of interest.

20. The method of claim 17 wherein the known compounds are associated with at least one compound parameter.

21. The method of claim 20 wherein the at least one compound parameter is a Log P value, a pKa value, a Log D value, a molecular weight value, a molar refractivity value, a number of compound hydrogen donors and acceptors value, a polar surface area value, a boiling point value or a molar volume value.
